(19) United States
(12) Plant Patent Application Publication
(10) Pub. No.: US 2022/0361390 P1
(43) Pub. Date: Nov. 10, 2022

(54) Anisidontea Plant Named 'IB710-1'

(50) ;

(71) Applicant: Plant Growers Australia Pty, Ltd., Wonga Park (AU)

(72) Inventor: Howard Bentley, Wonga Park (AU)

(73) Assignee: Plant Growers Australia Pty, Ltd., Wonga Park (AU)

(21) Appl. No.: 17/803,319

(22) Filed: May 9, 2022

Related U.S. Application Data
(60) Provisional application No. 63/185,702, filed on May 7, 2021.

(51) Int. Cl. A01H 6/60

(52) U.S. Cl. PLT/371

(57) ABSTRACT

A new and distinct Anisidontea capensis plant named ‘IB710-1’ which is characterized by a compact, densely foliaged growth, an abundance of large flowers, flowers with a dark red-purple center which become progressively lighter towards the apex and margins of the flower petals, and the stability of these characteristics from generation to generation.

Latin name of the genus and species: The Latin name of the genus and species of the novel variety disclosed herein is Anisidontea capensis.

Variety denomination: The inventive variety of Anisidontea capensis disclosed herein has been given the variety denomination ‘IB710-1’.

### BACKGROUND OF THE INVENTION

Parentage: ‘IB710-1’ is a seedling selection resulting from the controlled cross pollination of Anisodontea capensis ‘Dryspell Raspberry’ (not patented), the seed parent, with Anisodontea capensis ‘El Royo’ (not patented), the pollen parent. In the late summer of 2017 the above pollination occurred at the breeding site located in Wonga Park, Victoria, Australia. Seeds were collected from ‘Dryspell Raspberry’, sown, and seedlings were grown out to maturity, during the spring of 2017. One of the resulting plants was observed to exhibit medium to dense foliage, and shorter plant height, an abundance of large flowers, and red-purple flowers that fade to very light red-purple towards the outer region of the corolla. The seedling was isolated for further evaluation to confirm the distinctness and stability of the characteristics first observed. Upon confirmation of distinctness and stability, ‘IB710-1’ was selected for commercialization.

Asexual Reproduction: ‘IB710-1’ was first asexually reproduced by way of softwood stem cuttings in the spring of 2017 at the inventor's nursery in Wonga Park, Victoria, Australia. Through eight subsequent generations, the unique features of this cultivar have proven to be stable and true to type.

### SUMMARY OF THE INVENTION

The following characteristics have been repeatedly observed and represent the distinguishing characteristics of the new Anisidontea capensis plant, ‘IB710-1’. These traits, in combination, distinguish ‘IB710-1’ as a new and distinct cultivar.
1. ‘IB710-1’ exhibits a compact and densely-foliaged growth habit; and
2. ‘IB710-1’ exhibits an abundance of large flowers; and
3. ‘IB710-1’ exhibits red-purple flower petals which become progressively lighter red-purple towards the apex and margins; and
4. ‘IB710-1’ exhibits flower petals which are veined red-purple.

### BRIEF DESCRIPTION OF THE FIGURE

FIG. 1 illustrates, as nearly true as it is reasonably possible to make the same in color photographs of this type, an exemplary mature ‘IB710-1’ plant grown outdoors in Wonga Park, Victoria, Australia.

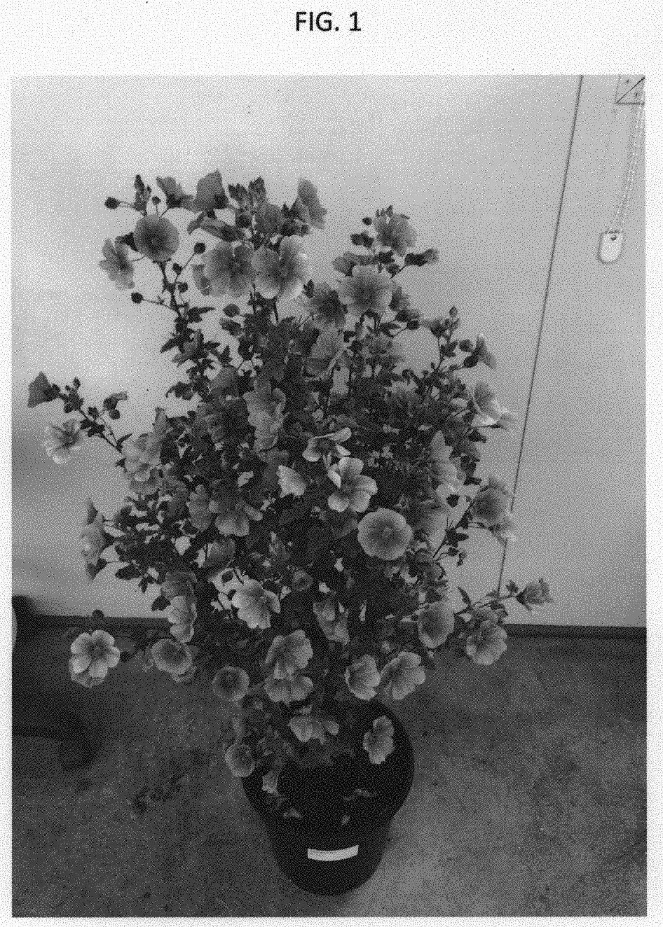

FIG. 2 illustrates, as nearly true as it is reasonably possible to make the same in color photographs of this type, exemplary flowers of a mature ‘IB710-1’ plant grown outdoors in Wonga Park, Victoria, Australia.

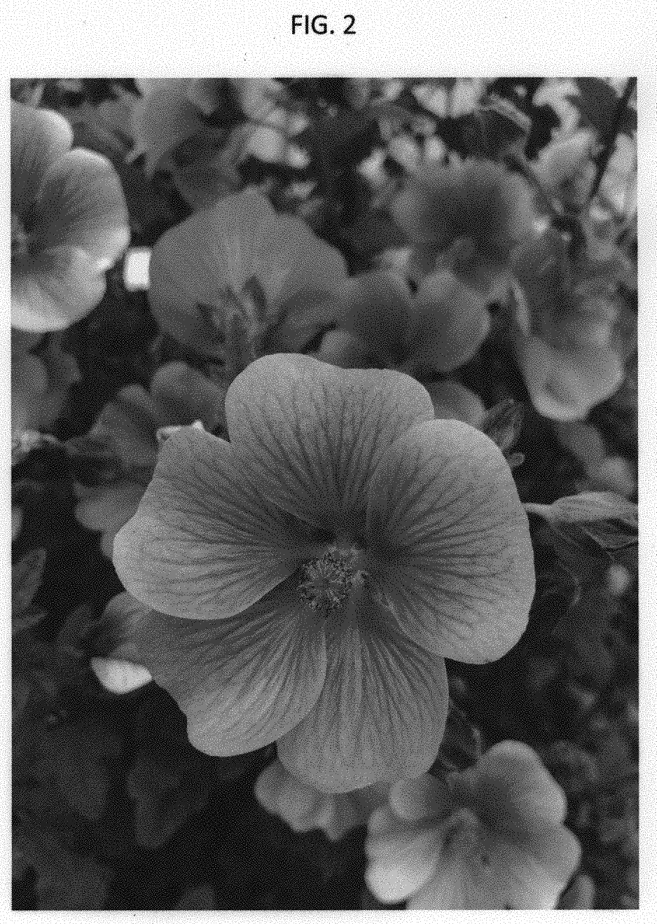

### BOTANICAL DESCRIPTION OF THE PLANT

A detailed botanical description of a new and distinct Anisidontea capensis plant known as ‘IB710-1’ is not yet available. Comparisons with the parents are provided below.

A botanical description of ‘IB710-1’ and comparisons with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and fruit: Botanical description is not yet available.

### COMPARISONS WITH THE PARENT PLANTS

Plants of the new cultivar ‘IB710-1’ differ from the seed parent, Anisodontea capensis ‘Dryspell Raspberry’ (not patented), in the following characteristics described in Table 1 below.

TABLE 1

| Characteristic | ‘IB710-1’ | ‘Dryspell Raspberry’ |
|---|---|---|
| Growth habit. | More compact and | Less compact and less |
| | densely foliaged. | densely foliage. |
| Foliage size. | Larger than ‘Dryspell | Smaller than ‘IB710-1’. |
| | Raspberry’. | |
| Flower size. | Larger than ‘Dryspell | Smaller than ‘IB710-1’. |
| | Raspberry’. | |
| General coloration | Red-purple at | Dark red-purple. |
| of the corolla. | the center of the | |
| | corolla and becoming | |
| | progressively lighter | |
| | red-purple towards | |
| | the apex and margins. | |

Plants of the new cultivar ‘IB710-1’ differ from the pollen parent and most similar variety known to the inventor, Anisodontea capensis ‘El Royo’ (not patented),in the following characteristics described in Table 2 below.

TABLE 2

| Characteristic | ‘IB710-1’ | ‘El Royo’ |
|---|---|---|
| Growth habit. | More densely foliaged | Less densely foliaged |
| | than ‘El Royo’. | than ‘IB710-1’. |
| Plant height. | Shorter than the | Taller than ‘IB710-1’. |
| | pollen ‘El Royo’. | |
| General coloration | A relatively darker | A lighter shade of |
| of the corolla. | shade of red-purple | red-purple at the center |
| | at the center of the | of the corolla, with |
| | corolla and becoming | a relatively darker |
| | progressively lighter | shade of red-purple |
| | red-purple towards | elsewhere on |
| | the apex and margins. | the flower petals. |

That which is claimed is:

1. A new and distinct variety of Anisidontea capensis plant named ‘IB710-1’, substantially as described and illustrated herein.
